(19) United States
(12) Patent Application Publication
(10) Pub. No.: US 2005/0031490 A1
(43) Pub. Date: Feb. 10, 2005

(54) Module for an analysis device, applicator as an exchange part of the analysis device and analysis device associated therewith

(76) Inventors: Walter Gumbrecht, Herzogenaurach (DE); Manfred Stanzel, Erlangen (DE); Manfred Wossler, Munchen (DE); Jorg Zapf, Munchen (DE)

Correspondence Address: STAAS & HALSEY LLP, SUITE 700, 1201 NEW YORK AVENUE, N.W., WASHINGTON, DC, 20005, US

(21) Appl. No.: 10/471,167

(22) PCT Filed: Mar. 8, 2002
(86) PCT No.: PCT/DE02/00836

(30) Foreign Application Priority Data
Mar. 9, 2001 (DE) 101 11 458.3

(51) Int. Cl. G01N 33/00

(52) U.S. Cl. 422/63; 422/58

(57) ABSTRACT

An analysis device that may be used in biochemical analyses includes a module in a first housing, including a chip support, a sensor chip and electrical contacts between the chip and the chip support. The chip is encapsulated so that the electrical contacts are insulated and the sensitive surface of the sensor chip remains accessible to a fluid to be tested. The module and the first housing form an exchangeable applicator or chip card with mocrofluidic components or functions and is inserted into a second housing that has an evaluation unit for reading and analyzing measured data.

### CROSS REFERENCE TO RELATED APPLICATIONS

This application is based on and hereby claims priority to German Application No. 101 11 458.3 filed on Mar. 9, 2001, the contents of which are hereby incorporated by reference. This application is related to ANALYSIS DEVICE, filed concurrently by Walter Gumbrecht and Manfred Stanzel and incorporated by reference herein.

### BACKGROUND OF THE INVENTION

1. Field of the Invention

The invention relates to a module for an analysis device, in particular for decentralized biochemical analytics, with a sensor chip in a first housing. In addition, the invention also relates to an applicator as an exchangeable part of the analysis device and to the associated analysis device.

2. Description of the Related Art

Microsensor technology and microsystems engineering have undergone a dramatic development in the last 20 years on the technological platform of microelectronics. All technical-scientific disciplines have made their respective contributions to this and created a broad spectrum of sensors and systems between physics and microbiology.

However, while physical concepts, such as for example pressure and acceleration sensors/systems, have gone through the process of implementation in terms of technical production and successful introduction on the market, most chemical-biological developments have not got beyond the laboratory trial stage. This has been significantly influenced by the fact that chemical-biological systems require microfluidic components which, by definition, are not compatible with microelectronics in the first place, since the classic microelectronic components are hermetically enclosed in a housing in order to avoid “material” contact with the surroundings. So it is that virtually all chemical-biological sensors/sensor systems are dependent on the development of a special housing technique.

There are a few cases in which microelectronic-compatible housing solutions have been developed to the stage of introduction on the market, for example ati-STAT Corporation, 303A College Road East, Princeton, N.J. 08540. Such a device is described in U.S. Pat. No. 5,096,669 A: one or more Si chips have sensitive areas with chemical sensors and contact areas for electrical connection to the reader. The chips are mounted in a housing in such a way that large parts of the chip areas are used for sealing a throughflow channel, and large contact areas for electrical contacting are accessible from outside the housing. Consequently, a large part of the valuable Si chip area is wasted. What is more, the electrical contacting in the housing is located on the same side as the sensitive areas of the chip, which makes it more difficult for the electrical contacting to be reliably separated from the fluidics.

Furthermore, in Dirks, G. et al. “Development of a disposable biosensor chipcard system”, Sens. Technol. Neth., Proc. Dutch Sens. Conf, 3rd (1988), pages 207 to 212, there is a description of a measuring system for biomedical applications in which a so-called chip card is made from a flat container with a number of cavities and a system of fluid channels, with an ISFET which serves as a sensor being introduced into the channel system. In the case of this system, it is in particular a matter of separately feeding a measuring fluid on the one hand and a calibrating or reagent fluid on the other hand to the sensor from separate containers. Furthermore, in the monograph by Langereis, G. R. “An integrated sensor system for monitoring washing process”, ISBN 90, there is a description of systems with sensors concerned with integrating in fluidic devices sensors which have their signals electrically tapped. On account of the high development and production costs for comparatively low numbers of units of chemical-biological systems, market penetration of these products is problematical.

### SUMMARY OF THE INVENTION

An object of the invention is therefore to propose improvements by which a successful introduction on the market appears possible in the case of the above devices.

In the case of a module according to the invention, it is particularly advantageous that the chip carrier is thin and has a thickness of <100 μm. With thicknesses of about 50 μm of metal in combination with about 100 μm of plastic, a considerable volume/material saving is obtained. On account of the thin formation of the chip carrier and suitable material, such as for example gold-coated copper layers, only small masses, and consequently low heat capacities, are obtained, so that, in combination with the good thermal conductivity of silicon and for example a copper/gold layer about 50 μm thick, a very good dynamic thermal behavior results. The processing of the chip carrier takes place on a strip which is transported from reel to reel (“reel to reel” process), it being advantageously possible for the electrical contacting points to be arranged on the rear side.

For the encapsulation of the chip carrier in the module, both materials known from microelectronics and materials with special properties, such as for example elastic polymers, may be used. Bonding wires, which form a flat loop, are present, it being possible for the contacts for the bonding wires to be arranged in the region of the corners of the chips.

Following mounting, wire bonding and encapsulation of the chips on the strip, the sensitive areas of the chips may be coated with chemical/biochemical substances, advantageously from the liquid phase, by a “reel to reel” technique. The encapsulation of the individual module in combination with the associated applicator produces particularly favorable properties.

With a module according to the invention, a system which is suitable in particular for decentralized applications can be created. With the compact first housing, the module realizes an applicator as a measuring unit which can be used in a decentralized manner. For carrying out the analysis and for reading out the measured values, the applicator can be introduced into a second housing with an evaluation unit.

In the case of the invention, the applicator with the first housing and the module integrated in it is advantageously formed in the manner of a chip card. Together with the second housing, such a chip card can form an analysis device which can be used in a variety of ways. In particular, an analysis device of this type can be used for the screening of body fluids, for example for decentralized blood gas measurements or saliva examinations. However, other applications in biochemical analytics can also be realized.

A further advantageous application possibility of the invention is the amplification of DNA/RNA (deoxyribonucleic acid/ribonucleic acid) samples by the exponential replication method with the so-called PCR (Polymer Chain Reaction), i.e. the so-called polymerase chain reaction method. For this purpose, the sample fluid must be cycled 20 to 40 times between two temperatures, typically between 40° C. and 95° C. In the case of this method, the speed of the cycling operations is decisive. As known in the art, the cooling process is speed-determining.

For practical purposes, a particularly advantageous embodiment, that is the chip card, comes into consideration as the applicator. In the case of the chip card, the Si chip is mounted on the carrier, which—as already mentioned—is made from a gold-coated copper layer only approximately 50 μm thick. This is the middle metal zone of known chip card modules, which is not used there for electrical contacting points in the card reader. This free zone can consequently be used in the card reader, which serves as an evaluation device, for contacting in particular a cooling element, for example a Peltier cooler, to the corresponding location of the chip card. On account of the placement of the 50 μm thick metallic contact with respect to the chip, an efficient heat transfer is consequently possible, so that a defined temperature can be set very quickly.

It is particularly advantageous in the case of the invention that the housing concept for realizing the microfluidics is based as much as possible on those of classic microelectronics. This creates the main prerequisites that allow modules with chemical-biological sensors or sensor systems of this type to have commercial success even in the case of relatively small numbers of units.

Apart from the latter advantages, in the case of the invention it is also taken into consideration that the chemical-biological sensor system can in particular also be used for once-only use, i.e. as a so-called disposable. Such systems are increasingly being adopted in practice.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 is a cross section through a chip module with wire bonding technology,

FIG. 2 is a cross section through a chip module with flip-chip technology,

FIG. 3 is a plan view of a chip card contacting zone with individual contacting points,

FIG. 4 is a plan view of the chip sensor with the sensitive area,

FIG. 4A is an enlarged plan view of the exposed sensitive area of the chip in FIG. 4 when the sensor is used for biochemical applications,

FIG. 5 is a cross section with a more detailed representation to scale of a chip card for the installation of a module with wire bonding technology,

FIG. 6 is a partial cross section corresponding to FIG. 5 for the installation of a module with flip-chip technology and reusable through-flow coupling,

FIG. 7 is a cross section of a combination of a module and an applicator for pushing into a reader and

FIG. 8 is a plan view from above and/or a cross section of the system illustrated in FIG. 7.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

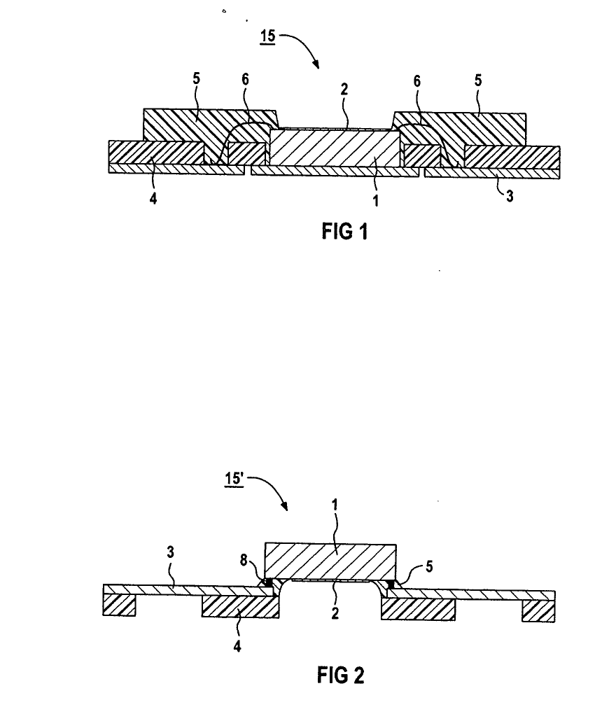

The drawings, in particular FIGS. 1 and 2, are partly described together.

Chip card technology is a known, widespread and extremely low-cost housing concept in microelectronics. In this case, a microsilicon chip, which has previously being ground thin to about 180 μm at wafer level, is adhesively attached to a carrier strip, which may be a gold-coated, pre-punched copper strip and is possibly reinforced with a strip of plastic. After standard wire bonding, the chip together with the wires is encapsulated in a polymer. A commercially obtainable standard plastic card (materials: PVC, PET, PC; dimensions: about 85×54×0.8 mm³) is milled out at a defined location to module size (about 13×12×0.4 mm³) for receiving the chip carrier module, so that once the module has been punched out of the carrier strip it can be adhesively bonded into the milled-out recess.

In FIG. 1, a chip module 15 with a sensor chip 1 in wire bonding technology is schematically represented. The module includes the actual chip 1 with a sensitive area 2 on the upper side, the chip 1 having been applied on the rear side of a carrier strip 3 of copper, which if appropriate is gold-coated. On the carrier strip 3 with area-like contact regions 3′, 3″, . . . there are elements 4 of plastic, which in particular mechanically hold together the insulated contacting areas 3′, 3″, . . . . Silicon microchips, such as for example microcontrollers or data memories, have in the past already been mass-produced in a similar formation, so that they are extremely inexpensive.

In the case of the chip module 15 constructed in FIG. 1, there is an encapsulation 5, in which bonding wires 6, 6′, . . . for the contacting of the chip 1 are cast in. While previously a closed surrounding of plastic covering the entire chip was provided by a so-called “glob top”, now the encapsulation 5 is formed flat with at least approximately a planar surface and opening, since the entire module 15 is to be introduced for example into a chip card as the housing.

In order to ensure complete wetting of the sensitive chip area 2 under operating conditions of the analysis device, i.e. to avoid the inclusion of air bubbles during filling with fluids, it is important that the ratio of the height of the encapsulation above the upper edge of the chip 1 to the diameter of the sensitive area of the chip 1 does not exceed approximately 1:5 and is typically less than 200 μm. As revealed by FIG. 5, which is to scale, 100 μm is an advantageous height for the encapsulation above the upper edge of the chip 1. In order to seal the flow channels, for example the inflow and outflow channels 12, 13 in FIG. 5, reliably with respect to the first housing, the encapsulation 5 must have a defined lateral extent. A widening of the lateral extent of the encapsulation is necessary inter alia if the inflow and outflow are to lie outside the sensitive area of the chip 1, in order for example to avoid disturbing influences of an inhomogeneous flow of the fluids. The inflow and outflow then meet the sensor module in the region of the encapsulation and can be reliably sealed there.

In a particular embodiment, the encapsulation 5 has a diameter of 10 mm and a clearance for the sensitive area 2 of the chip of 3 mm. In combination with the ratio described above of the height of the encapsulation to the diameter of the sensitive area 2, a uniform flow of the fluids onto the sensitive area 2, i.e. parallel to the sensitive area of the chip, is made possible.

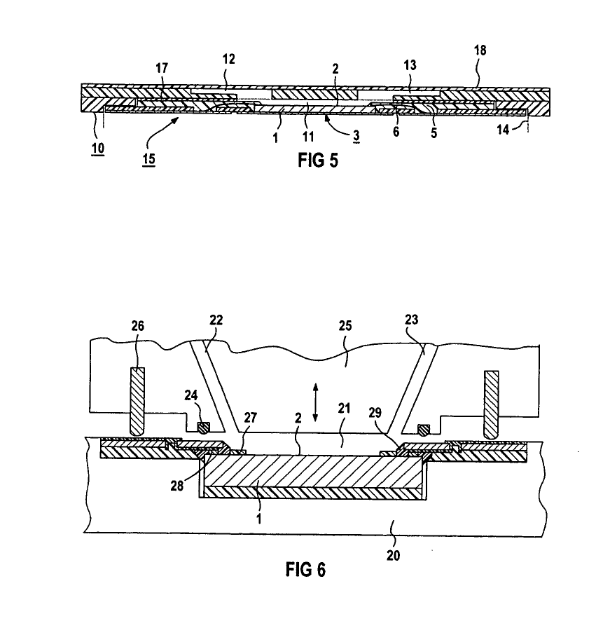

The sensitive area 2 of the chip is preferably formed in a round manner. The delimitation of the sensitive area 2 with respect to the encapsulation 5 can be realized for example by a photostructured polymer ring, as described further below in FIG. 6 as a PI (polyimide) ring 27.

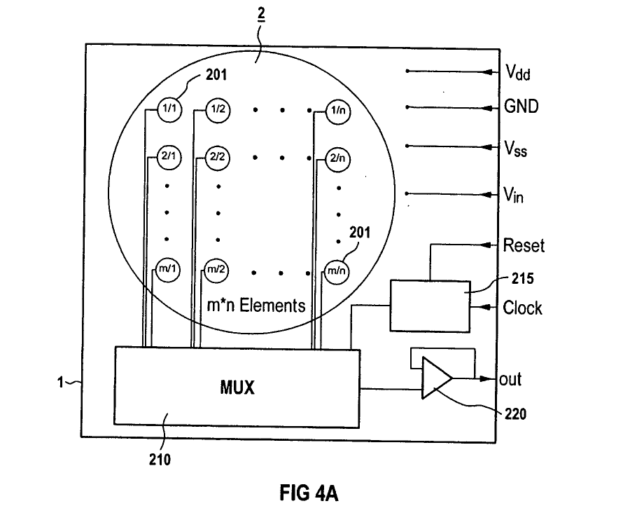

In order to maximize the ratio of the sensitive area 2 to the overall area of the chip 1, the form of the chip 1 is preferably approximately or exactly square, the electrical contacts of the chip 1 as so-called bonding pads 2′ to 2^{VII} being located in the region of the chip corners, so that the sensitive area can be made to extend up to the chip edges, which is revealed in FIG. 4. With a thickness of the metallization of the carrier strip of 50 μm, a chip thickness of 180 μm and height of the encapsulation above the chip 1 of 100 μm, an overall thickness of the module of approximately 330 μm is obtained. Consequently, the known chip module structures and dimensions from microelectronics are transferred to biochemical analytics, which is not a trivial matter on account of the necessary coupling of the fluidics.

In the case of an alternative to FIG. 1, according to FIG. 2 the chip 1 is oriented with its sensitive area 2 downward. The sensor chip 1 is arranged in so-called flip-chip technology with a number of bump-like contacts 8, 8′, . . . on the carrier strip 3 with its contact regions 3^{I}, 3^{II}, . . . , 3^{VIII}, the carrier strip formed of copper, if appropriate with a gold coating, in a form corresponding to FIG. 1. Insulating elements 4 are in turn present as mechanical connections of electrically insulating plastic, a clearance for the sensitive area 2 of the sensor chip 1 being present. Altogether, a chip module 15′ is formed in FIG. 2.

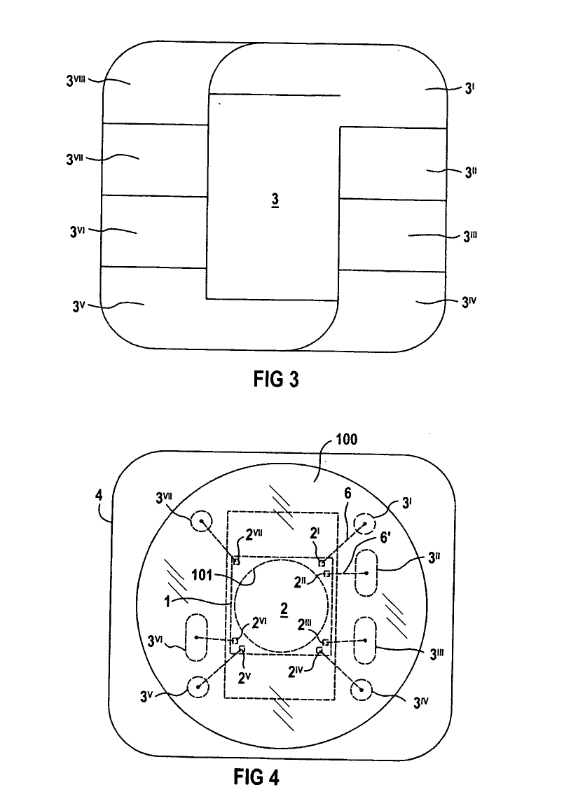

The operating principle of the chip module 15 or 15′, and in particular of the actual chip 1, is illustrated by the views from two sides of the module on the basis of FIGS. 3 and 4. On the electrical contact side 3, i.e. the rear side, of the module 15 with the sensor chip 1, contacting zones 3^{I}, . . . , 3^{VIII} can be seen as individual terminals, which correspond to the customary contacting points for chips which can be integrated into a card. On the sensitive side 2 of the chip 1, according to FIG. 4 the wire bonds 6, 6′, . . . of the bonding pads 2^{I} to 2^{VII} run from the corners of the chip 1 to the contacts of the contacting zones 3^{I}, . . . 3^{VIII}. It is evident that here specifically there are seven contacts 2^{I}, . . . 2^{VII} on the chip area 2, which is sufficient for many applications and is described below for an example.

In FIG. 4A, a multiplicity of microcavities 200 for carrying out biochemical analyses are arranged on the sensitive area 2 of the chip 1. Such a system is described for example in the earlier German patent application with the application number 100 58 394.6-52, to which reference is expressly made, and serves for carrying out biochemical measurements, for example DNA analysis. There are m×n elements arranged in the form of an array as a multiplicity of cavities 200 in the form of rows and columns. The important aspect of this is that biochemical reactions or measurements can take place simultaneously in the individual cavities 200 on the sensitive surface of the single chip 1, without reactions from a first cavity 200 being able to disturb a second cavity 200′ when substances are added.

Since in the case of a system according to FIGS. 4 and 4A the electrochemical reactions electrically influenced or takes place by inquiring electrical signals, discrete electrical contacting points, which are designated by 3^{I} to 3^{VII}, have been attached on the chip 1 with a sensitive surface 2 or the individual sensitive elements 200. The contacting points form inputs for the electrical measuring circuit. For example there are two supply voltage inputs V_{dd}, Vₛₛ, an input GND for ground potential, an input for a clock signal, an input Vᵢₙ for a control voltage and an input for a reset signal. Furthermore, a multiplexer 210, a “Gray counter & decoder” 215 and an amplifier 220 are integrated on the chip 1 by a standard silicon technique. The measuring signal is sensed at the ‘out’ output, with a multiplex signal which is read out for example at a frequency of 10 kHz being obtained in the case of an array system with the multiplicity of cavities as m×n individual sensors.

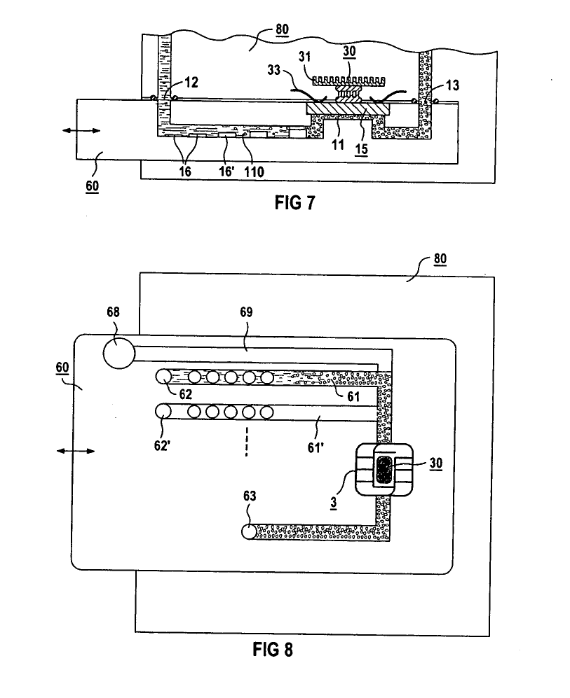

The multiplex signal output on a single ‘out’ line includes a pattern of discrete voltage values, from which the signals of the individual sensor are obtained by a demultiplexer in an evaluation device. The demultiplexer, not represented in FIG. 4A, is arranged for example in the housing 80 of FIG. 7 or FIG. 8.

In another system, instead of a multiplicity of identical sensors, such as the m×n cavities 200 corresponding to FIG. 4A, there may also be discrete sensors. Specifically for applications in biomedical technology, such sensors may be, for example, sensors for pO₂ and pCO₂.

Further sensors may also be combined with these. The eight contact zones available in the case of the system according to FIG. 3 are generally adequate for signal supply and signal removal. By dividing the electrical contacting and fluid access between opposite sides of the sensor module 15, by contrast with U.S. Pat. No. 5,096,669 A a reliable separation of the electrical contacting from the fluidics is ensured. Furthermore, unproblematical fluid access to the sensor module is made possible. A circular planar surface 100 of the encapsulation 5 of plastic with an advantageously inner round clearance 101 on the chip 1 achieves the effect of reliable insulation of the wire bonding contacting points 6, 6′ and equally keeps the sensitive chip area 2 centrally free.

The production of the sensor modules takes place in a so-called “reel to reel” process as known technology on a flexible basic body. In the “reel to reel” process, a carrier strip is processed, i.e. the operations a) adhesive chip attachment, b) wire bonding/flip-chip, c) encapsulation are processed in an automated manner from film reel to film reel—which in mass production can take place on a conveyor belt—up to the finished module. Subsequently, the modules are punched out and installed in a close-fitting manner into the “first housings”.

In FIGS. 5 and 6, the two alternative systems of modules introduced in a first housing are represented, with wire-bonding technology on the one hand and flip-chip technology on the other hand. In both cases, the system respectively includes substantially a standard plastic card 10 or 20 with microfluidic components and functions, which will be discussed in more detail further below. Especially the card 10 may have additional layers 18, for example an adhesive film or the like, with which the entire unit is sealed against environmental influences.

In the card 10 according to FIG. 5, a microchannel 11 and inflow/outflow channels 12 and 13 are present as microfluidic components, which serve inter alia for transporting substances and/or reagents. What is important is a clearance 14 in the housing 10, into which the chip module 15 according to FIG. 1 or FIG. 2 is introduced in suitable positioning. The clearance 14 must be adapted to the encapsulation 5 of the chip 1. In this case, a radial symmetry with an axis perpendicular to the active area of the chip 1 and/or a planar encapsulation parallel to the active area of the chip 1 may be advantageous.

During the mounting of the module 15 into the clearance 14 of the first housing 10, a fluid-tight connection must be ensured between the surface of the encapsulation 5 and a layer 19 of a material which carries microfluidic components, such as the inlet 12 and outlet 13. This may be achieved by adding auxiliary means such as adhesives or double-sided adhesive tapes 17. In a particularly advantageous embodiment, it is possible to dispense with the auxiliary means by using an elastic encapsulating material 5. During the operation of the analysis device, the elastic encapsulation 5 is pressed onto the material of the layer 19 which is carrying the microfluidic elements of the first housing 10, so that the channel 11 with the inlet 12 and the outlet 13 are sealed. The pressing may take place for example by an actuator in the second housing.

The entire chip module 15 or 15′ corresponding to the alternatives according to FIG. 1 or FIG. 2, including the silicon chip 1 with the sensitive area 2, is consequently inserted into the basic body, in particular the card body 10 in FIG. 5, in such a way that the system is adequately sealed with respect to the outside, allows an inflow or entry of substances to be analyzed and only the active area of the chip 1 can come into interaction with the substances to be analyzed. In order to ensure complete wetting of the sensitive chip area 2 during operation, i.e. to avoid the inclusion of air bubbles, in particular in the channel 11, it is important that the ratio of the height of the gap in the microchannel 11 between the chip 1 and the layer 19 which is carrying the channels with inlets and outlets 12, 13 to the diameter of the sensitive area 2 of the chip 1 is less than 1:5 or the gap 11 is typically smaller than 200 μm.

The specified gap of smaller than 200 μm is of advantage in the case of diffusion-controlled reactions, for example DNA hybridizing, on the sensitive area 2 of the chip 1. By making the co-reactants, which are for example dissolved in the sample fluid, flow in a thin layer over the reactive, sensitive chip area 2, they can be offered in higher concentration on the surface of the chip 1 in comparison with diffusion alone, which leads to speeding up of the reaction.

Represented in FIG. 6 as an alternative to FIG. 5 is a system which includes a card body 20 without internal fluidic components and in this case also without electrical functions. The chip 1 is contacted onto the card body 20 with the sensitive area 2 oriented upward.

As a departure from FIG. 5, in FIG. 6 a partially “reusable” flow cell is used. The electrical inquiry and also the supply and removal of sample fluids takes place from the outside. In the same way, of course, the chip module 15 according to FIG. 1 may also be operated with a reusable flow cell, but then however with advantageous electrical contacting on the rear side.

In FIG. 6, the card body 20 forms the first housing, with the measuring and analyzing function being realized in the upper part as a second housing. The fluidic and electrical components can be found in the upper part.

In FIG. 6, the upper part 25, which is the carrier of inflow and outflow channels 22 and 23, is mounted on the basic body 20, which together with the module realizes the chip card as an applicator, in such a way that a so-called contact head is formed. The upper part 25 as the contact head has resiliently mountable electrical contacts 26 and sealing means, such as for example a sealing ring 24, are also present. The sealing ring 24 serves for ensuring the tightness of the seal in the fluidic region 21 between the upper part and the sensitive area 2 of the chip 1 with the resiliently mounted contacts 26 of the contact head 25 for the electrical contacting through the chip 1.

In the applicator 20 of FIG. 6, by analogy with FIG. 5, the module according to FIG. 2 has been fitted with the silicon chip 1, the sensitive chip area 2 again being shown upward even with the flip-chip technology applied here—by contrast with FIG. 2, for the purpose of illustrating the principle of flip-chip technology. The sensor chip 1 including the carrier has in this case been fitted in the card body 20.

Further auxiliary components of flip-chip technology are present for the latter purpose, such as for example a PI ring 27, a so-called underfill 29 and a so-called bump 28, for sealing and maintaining the dimensional stability of the chip position. These auxiliary components have proven successful in semiconductor technology and ensure the required quality during the manufacture of the sensor chips, in particular when the fluidics on the sensor area are to be managed.

The essential aspect in the case of FIG. 6 in the present connection is that the separate upper part 21 only has to be mounted onto the basic body 20 for measurement, and then, in this applied state, equally ensures on the one hand the fluidic connection and on the other hand the electrical contacting at the existing through-contacting holes.

The card 10 according to FIG. 5 and the body 20 according to FIG. 6 consequently form in each case a separately exchangeable, flat applicator with a first housings for the respective measuring modules. For analysis and for reading out the measuring signals, these applicators with the first housing are pushed into a second housing in each case, which is for example part of a stationary measuring and analysis device or else may be a portable device for measuring activities in changing locations.

Represented in FIGS. 7 and 8 is an applicator, having a sensor module 15 and a first housing 60, which has been pushed into a second housing 80 for carrying out the measurement and for reading out the measured values. The sensor module 15, described in detail on the basis of FIGS. 1, 4, 4A, has its functional area facing a fluid channel 11, into which measuring and reagent solutions are introduced via a channel 110. The reagent solution is produced in situ from pre-portioned solid reagents 16, 16′, 16″ with a solvent fed in via an inlet 12. The measuring and reagent solutions pass via an outlet 13 to the second housing 80 for the purpose of disposal.

The latter system is substantially the subject of a parallel application with the same priority date (German patent application number 101 11 457.5-52 of Mar. 9, 2001), to the disclosure of which reference is expressly made.

In FIG. 7, a Peltier element 30 for thermostatic control, in particular cooling, of the chip area is assigned to the sensor module 15 with associated contacts on the rear side in the second housing 80, so that it is possible to operate at defined temperatures or rapid heat removal is ensured in cooling processes from high temperatures, for example 90° C., to lower temperatures, for example 30° C. On account of the materials with very good heat conductivity, silicon and copper/gold, but also the low layer thicknesses (about 180 μm of silicon; 50 μm of copper/gold), an outstanding heat transfer is ensured. For the Peltier element 30, a cooling plate 31 is provided and, furthermore, electrical clamping contacts 33 are provided for the reading out of the chip information. By pressing the Peltier element 30 against the sensor module 15, apart from improving the heat transfer, the sealing described in detail above of an elastic encapsulation 5 of the module 15 to the material of the layer 19 carrying the microfluidic channels can take place.

The latter system can be used advantageously for the amplification of DNA/RNA (deoxyribonucleic acid/ribonucleic acid) by an exponential replication method, the so-called PCR (Polymer Chain Reaction). For this purpose, the DNA/RNA sample and required reagents, such as for example nucleotide triphosphates, primary DNA/RNA and polymerase/polymerase+reverse transcriptase in buffer solution are fed to the sensitive area of the sensor chip via the microfluidic channels. The immobilization of the DNA/RNA sample on the sensitive area of the chip is particularly advantageous here. This can take place for example by hybridizing on complementary capture DNA, which is bound on the chip, for example in the form of arrays. The reaction space, i.e. the space over the sensitive area of the chip with a height of up to several hundred μm, is then cycled approximately 20 to 40 times between two temperatures, typically between 40° C. and 95° C. In the case of this system, the entire DNA/RNA replication process can be carried out in a few minutes.

According to FIG. 8, a first reagent channel 61, which is connected to a water inlet 62, is present for the latter purpose in the first housing 60. Furthermore, there is a second reagent channel 61′, which runs parallel to the first reagent channel 61 and, by contrast with the reagent channel 61, is not filled in the representation of FIG. 7. The second reagent channel 61′ can be connected to a second water inlet 62′. Further parallel-connected reagent channels 61″, . . . may be provided, with water inlets 62″, . . . , which are respectively parallel-connected, so that altogether n reagent channels and n water inlets are formed. Furthermore, there is an input port 68 for the fluid which is to be examined, for which the measurement sample is transported via a channel 69 to the sensor module 15, without previously having to come into contact with the reagent fluid. Finally, an outlet 63 is provided, via which the fluid is discharged after flowing past the sensitive area 2 of the sensor module 15.

Alternatively, the used fluids may remain in a corresponding volume, for example by widening of the channel or lengthening of the channel in the form of a meander, of the first housing. In the reader of the second housing 80, a water distribution system with valves is provided.

The described example of an analysis device with chip cards which can be pushed into a reader as measuring applicators consequently makes use of the main components and of previous chip card technology. For the operating principle of a chip card with combined electrical and fluidic components, the following main, non-trivial changes or additional features are provided:

A modified encapsulation of the chip and of the electrical contacts via bonding wires ensures that only the chemical-biologically active area of the chip remains free from the encapsulation.

The modified encapsulation of the sensor chip and of the associated bonding wires has a defined geometry.

The encapsulation has a defined thickness, a defined lateral extent and also an at least approximately planar and/or radially symmetrical surface for the exact insertion of the sensor chip into a chip card.

To sum up, the following should also be emphasized in addition to the above examples with respect to the use of chip card technology in chemical-biological measurement: in all the embodiments, the configuration of the system including the chip card with the functional volume takes place in such a way that microfluidic components and functions are integrated in the interior and/or on the surface of the card. This makes it possible for liquids or gases to enter the chip card and be transported in the interior or on the surface of the chip card and be available in the region of the silicon chip of the active area of the chip. This is where the measurement takes place, after which the liquids or gases in the region of the silicon chip can subsequently be carried away from the active area of the chip and leave the chip card. If appropriate, substances can be stored in the interior or on the surface of the chip card or remain there after use.

An important aspect is the clearance in the chip card for receiving the chip module in such a way that a reliable microfluidic connection is made possible between fluid channels of the plastic card and the active, i.e. sensitive, area of the chip and no external influences can disturb the measurement.

Dependent on the required position of the microfluidic components, the chip card may include one or more components or layers, which are joined together by known connecting methods, such as adhesive bonding, welding, laminating or the like.

The components for the microfluidic functions may be produced by a wide variety of methods, such as milling, punching, stamping, injection-molding, laser ablation or the like.

On account of certain requirements, for example with respect to the chemical resistance or the thermal endurance, the applicator itself may be made of a wide variety of materials and consequently be adapted to the requirements in the particular instance.

It is possible to the greatest extent to rely for this purpose on the know-how of card technology.

This consequently provides an analysis device which, apart from in biochemical analytics, can also be used in a variety of ways, in particular for use in medical diagnostics, forensics, for food monitoring and for environmental measuring technology. The decentralized use of the applicator and reader allows time-saving low-cost examination on the spot, in particular in clinics and doctors' own practices, of for example blood, liquor, saliva and smears, for example for viruses of infectious diseases. This may include, if necessary, not only simple typing of the germs, but also for example the determination of any resistances to antibiotics, which significantly improves the quality of the therapy and consequently can reduce the duration and cost of the illness.

Apart from the diagnosis of infectious diseases, the diagnosis system is for example also suitable in medicine for blood gas/blood electrolyte analysis, for therapy control, for early detection of cancer and for the determination of genetic predispositions.

For all the intended uses specified, the applicator may be formed as an autonomous unit, in which a voltage source, simplified evaluation electronics and a display are present in the applicator housing.

The invention has been described in detail with particular reference to preferred embodiments thereof and examples, but it will be understood that variations and modifications can be effected within the spirit and scope of the invention.

1-34. (canceled)

35. A module for a decentralized biochemical analysis device, comprising:
a sensor chip having a sensitive area and electrical contacts; and
a carrier having contact zones associated with the electrical contacts of said sensor chip and an encapsulation with contact connection between the contact zones and the electrical contacts of said sensor chip to provide electrical access from outside said module, the encapsulation allowing access by a fluid to the sensitive area of said sensor chip.

36. The module as claimed in claim 35, wherein a ratio of height of the encapsulation above an upper edge of said sensor chip to a largest diameter of the sensitive area of said sensor chip is less than 1 to 5.

37. The module as claimed in claim 35, wherein the encapsulation of said sensor chip has a defined lateral extent to seal fluidic inflow and outflow.

38. The module as claimed in claim 35, wherein the encapsulation includes an elastic material, whereby the fluidic inflow and fluid outflow can be sealed without aid of further means.

39. The module as claimed in claim 35, wherein the electrical contacts of said sensor chip are bonding pads in corners of said sensor chip.

40. The module as claimed in claim 36, wherein the encapsulation has at least one of a substantially planar surface and a radially symmetrical surface.

41. The module as claimed in claim 40, wherein said module is a chip card.

42. The module as claimed in claim 35, wherein said carrier is a metallic carrier strip having a thickness of less than 100 μm and the contact zones are plastic-reinforced metal contacts.

43. The module as claimed in claim 42, wherein said sensor chip is mounted on the metallic carrier strip by wire bonding.

44. The module as claimed in claim 42, wherein said sensor chip is mounted on the carrier strip as a flip-chip.

45. An applicator as an exchangeable part of an analysis device, comprising:
a first housing, including
a module, including
a sensor chip having a sensitive area and electrical contacts; and
a carrier having contact zones associated with the electrical contacts of said sensor chip and an encapsulation with contact connection between the contact zones and the electrical contacts of said sensor chip to provide electrical access from outside said module, the encapsulation allowing access by a fluid to the sensitive area of said sensor chip; and
means for inflow and outflow of fluids to the sensitive area of said sensor chip.

46. The applicator as claimed in claim 45, wherein said first housing includes a gap filled with fluids during operation over the sensitive area of said sensor chip and a ratio of a height of the gap to a largest diameter of the sensitive area of said sensor chip is less than 1 to 5.

47. The applicator as claimed in claim 45, wherein said first housing includes a gap of less than 200 μm filled with fluids during functional operation over the sensitive area of said sensor chip.

48. The applicator as claimed in claim 45, wherein said module and said first housing are formed as a chip card with microfluidic components and functions integrated in therein.

49. The applicator as claimed in claim 45, wherein said sensor chip is provided with microfluidic components that feed and carry away at least one of liquids and gases respectively to and from the sensitive area of said sensor chip.

50. The applicator as claimed in claim 45, further comprising storage for at least one of solids, liquids and gases.

51. The applicator as claimed in claim 50, wherein said means for inflow and outflow of fluids include a microfluidic connection between said storage and the sensitive area of said sensor chip.

52. The applicator as claimed in claim 51, wherein said first housing is a card having at least one layer.

53. The applicator as claimed in claim 52, wherein said first housing is a card made of multiple materials.

54. The applicator as claimed in claim 52, wherein said first housing further includes an integrated voltage source, evaluation electronics and display.

55. An analysis device, comprising:
an applicator for decentralized measurements, including a first housing having an interior and a surface, including
a module, including
a sensor chip having a sensitive area and electrical contacts; and
a carrier having contact zones associated with the electrical contacts of said sensor chip and an encapsulation with contact connection between the contact zones and the electrical contacts of said sensor chip to provide electrical access from outside said module, the encapsulation allowing access by a fluid to the sensitive area of said sensor chip; and
means for inflow and outflow of at least one of liquids and gases to the sensitive area of said sensor chip via one of the interior and on the surface of said first housing; and
a second housing, including an evaluation unit, into which said applicator can be introduced, to perform analysis and read out measurement data.

56. The analysis device as claimed in claim 55,
wherein said applicator is a chip card; and
wherein said second housing carries out the analysis and reads out the measurement data after said chip card is pushed into said second housing.

57. The analysis device as claimed in claim 56, wherein when said second housing carries out the analysis and reads out the measurement data, at least one of liquids and gases are transferred between said applicator and said second housing.

58. The analysis device as claimed in claim 55,
wherein said first housing includes clearances,
wherein the encapsulation includes an elastic material, and
wherein said second housing further comprises means for pressing the elastic encapsulation of said module against the clearances in said first housing.

59. The analysis device as claimed in claim 58, further comprising temperature control means for setting a defined temperature at the sensitive area of said sensor chip by cooling.

60. The analysis device as claimed in claim 59, wherein said temperature control means comprises a Peltier element in said second housing for the sensor chip.

61. The analysis device as claimed in claim 55, wherein the analysis device performs biochemical analytics.

62. The analysis device as claimed in claim 61, wherein the analysis device performs DNA analysis.

63. The analysis device as claimed in claim 61, wherein the analysis device uses a Polymer Chain Reaction and said analysis device speeds cooling during the Polymer Chain Reaction.

64. The analysis device as claimed in claim 55, wherein the analysis device performs food monitoring.

65. The analysis device as claimed in claim 55, wherein the analysis device performs environmental measuring.

66. The analysis device as claimed in claim 55, wherein the analysis device performs forensics analysis.

67. The analysis device as claimed in claim 55, wherein the analysis device performs medical diagnostics.

68. The analysis device as claimed in claim 65, wherein the analysis device performs blood gas/blood electrolyte analysis.
